(19) United States
(12) Patent Application Publication
(10) Pub. No.: US 2007/0276354 A1
(43) Pub. Date: Nov. 29, 2007

(54) Introducer Sheath and Method for Making

(75) Inventor: Thomas A. Osborne, Bloomington, IN (US)

Correspondence Address: BRINKS HOFER GILSON & LIONE/INDY/COOK, ONE INDIANA SQUARE, SUITE 1600, INDIANAPOLIS, IN, 46204-2033, US

(73) Assignee: Cook Incorporated, Bloomington, IN (US)

(21) Appl. No.: 10/581,330

(22) PCT Filed: Jul. 15, 2005
(86) PCT No.: PCT/US05/25188

§ 371 Date: Mar. 7, 2007

Related U.S. Application Data
(60) Provisional application No. 60/589,758, filed on Jul. 21, 2004.

(51) Int. Cl. A61M 25/16

(52) U.S. Cl. 604/527

(57) ABSTRACT

An introducer sheath and a method of manufacturing an introducer sheath. The introducer comprises a first polymeric sleeve having a first striped extrusion arranged in a generally helical pattern along the first sleeve. A second polymeric sleeve is positioned over and bonded to the first polymeric sleeve, the second polymeric sleeve comprising a second striped extrusion that is arranged in a generally helical pattern along the second sleeve. The first and second polymeric sleeves are axially aligned such that the second striped extrusion is superposed over the first striped extrusion to define a generally braid-like configuration. The introducer sheath can optionally include an inner liner disposed within a lumen of the first polymeric sleeve, and/or a coil fitted over the inner liner, such that the first polymeric sleeve is bonded to the inner liner between turns of the coil.

### BACKGROUND

1. Technical Field

The present invention relates to an apparatus, such as an introducer sheath, for use in the placement of a medical interventional device, and to a method for making the apparatus.

2. Background Information

Percutaneous entry devices, referred to herein as “introducer sheaths”, are typically used to introduce medical interventional devices, such as balloon angioplasty catheters and stents, into the vasculature. Such introducer sheaths are typically thin-walled tubular devices that are fitted to an inner dilator for percutaneous placement over a wire guide. Current introducer sheaths are often extruded from compositions such as PTFE or PFEP. Other introducer sheaths are typically formed as composite constructions consisting of an inner liner formed of a low friction, lubricous material such as PTFE, an intermediate reinforcing layer consisting of a braid or a coil, and an outer layer formed of a thermoplastic compound such as a polyamide, polyethylene, polyurethane, and the like.

Prior art introducer sheaths formed as composite constructions that incorporate a braid as the intermediate reinforcing layer generally do so to enhance the torqueability of the device. Braids are known to enhance torque control, which enhanced control assists the physician when directing a preformed tip into branch arteries and vessels. This action allows the accurate placement of stents and balloon angioplasty catheters in precise, distal locations. Prior art introducer sheaths that utilize a coil as an intermediate layer generally do so to enhance the kink resistance of the device. This allows the physician to manipulate the guide catheter or sheath external to the patient without kinking, and to conform to tortuous anatomy within the patient. If an introducer sheath kinks, the lumen size and the ability of the sheath to freely deliver other devices, such as stents, will normally be compromised.

Multi-layer introducer sheaths such as those described above are generally constructed by placing the inner liner material over a mandrel. The braid or coil is then placed over the outer surface of the inner liner. The outer thermoplastic material is then placed over the braid or coil. A heat shrinkable sleeve is placed over the assembly, and the assembly is heated or baked in an oven. This causes the thermoplastic outer layer to melt and flow between the wires of the braid or coil, such that it bonds to the inner liner. When the assembly is cooled, the heat shrink sleeve is slit and peeled off the thermoplastic layer, and the mandrel is pulled out of the inner liner. The result is a thin-walled multi-layer tube suitable for use as a guide catheter or vascular sheath. Such sheaths are further discussed, e.g., in U.S. Pat. No. 5,380,304, incorporated by reference herein.

Attempts have been made to construct introducer sheaths having both a braid and a wire coil as an intermediate layer, in order to achieve both enhanced torqueability and kink resistance. To date, however, the resulting sheaths exhibit shortcomings. For example, utilizing both reinforcements in an intermediate layer results in a structure that may be too thick-walled for some proposed uses, hi addition, the wire or monofilament layers are susceptible to interfering with each other, in which case the resulting device would have neither good torqueability nor good kink resistance.

It is generally desired that introducer sheaths and guide catheters have a very thin wall, e.g., 0.010 inch (0.254 mm) or less, to allow the entry site into the vessel to be as small as possible. If the sheath is much larger than about 0.010 inch (0.254 mm), the entry site may be of a size to cause damage to the vessel wall, and/or it may cause difficulties in the manipulation of the sheath or catheter through the anatomy. In addition, if a combined braid and coil layer is provided, this layer is difficult to over coat properly with the thermoplastic layer. In order for the introducer sheath to be properly constructed such that the outer layer is securely bonded to the inner liner, it is important that the outer layer be able to flow through the braid and coil wire layer during melting of the outer layer. If the melted outer layer cannot flow through the wires of both the braid and sheath, there may be insufficient bonding of the outer layer to the inner liner. This may result in the dislodgement of one layer from the other during use of the device.

An example of a prior art coil reinforced sheath is the FLEXOR® sheath, available from Cook Incorporated, of Bloomington Indiana. The FLEXORS sheath is widely used for the placement of stents and other devices, and has been found to function very well in such use. This sheath includes a coil reinforcement, and exhibits a high level of kink resistance. However, once the tip of this sheath has been placed in the vasculature, the torque control of the sheath can be less than optimal, and it can be difficult to rotationally control the direction of the tip in some applications.

It is desired to provide an introducer sheath that overcomes the problems associated with prior art sheaths. More particularly, it is desired to provide an introducer sheath that has a low profile, and has high level of torqueability during normal usage.

### BRIEF SUMMARY

The present invention addresses the problems of the prior art by providing a low profile introducer sheath having enhanced torqueability, and a method for making the introducer sheath.

In one form thereof, the invention comprises an introducer sheath. The introducer sheath comprises a first polymeric sleeve having a first striped extrusion that is arranged in a generally helical pattern along the first sleeve. A second polymeric sleeve is positioned over and bonded to the first polymeric sleeve, the second polymeric sleeve comprising a second striped extrusion that is arranged in a generally helical pattern along the second sleeve. The first and second polymeric sleeves are axially aligned such that the second striped extrusion is superposed over the first striped extrusion to define a generally braid-like configuration. If desired, the introducer sheath can also include an inner liner disposed within a lumen of the first polymeric sleeve, and a coil fitted over the inner liner. The first polymeric sleeve is bonded to the inner liner between turns of the coil.

In another form thereof, the invention comprises a method of manufacturing an introducer sheath. A first polymeric sleeve is positioned over a mandrel, the first polymeric sleeve comprising a first striped extrusion arranged in a generally helical pattern along the first sleeve. A second polymeric sleeve is positioned over the first sleeve, the second polymeric sleeve comprising a second striped extrusion arranged in a generally helical pattern along the second sleeve. The first and second polymeric sleeves are axially aligned such that the second striped extrusion is superposed over the first striped extrusion to define a generally braid-like configuration. The first and second polymeric sleeves are then bonded together by heating. Optionally, the sheath can also be manufactured to include an inner liner and/or a coil.

### BRIEF DESCRIPTION OF THE DRAWINGS

FIG. 1 depicts an introducer sheath, shown in combination with a dilator and a connector valve;

FIG. 2 is an elevational view of a sleeve according to an embodiment of the present invention that incorporates numerous stripes of a material for imparting torque control;

FIG. 3 is a sectional view of the sleeve of FIG. 2, taken along line 3-3 of FIG. 2;

FIG. 4 is an elevational view of two twisted stripe extrusions, one inside the other;

FIG. 5 shows a sectional view of the stripe extrusions of FIG. 4, taken along line 5-5 of FIG. 4;

FIG. 6 illustrates a method for forming the introducer sheath; and

FIG. 7 illustrates an alternative embodiment of a method for forming an introducer sheath.

### DETAILED DESCRIPTION OF THE PREFERRED EMBODIMENTS

For the purposes of promoting an understanding of the principles of the invention, reference will now be made to the embodiments illustrated in the drawings, and specific language will be used to describe the same. It should nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates.

In the following discussion, the terms “proximal” and “distal” will be used to describe the opposing axial ends of the introducer sheath, as well as the axial ends of various component features. The term “proximal” is used in its conventional sense to refer to the end of the sheath (or component thereof) that is closest to the operator during use of the sheath. The term “distal” is used in its conventional sense to refer to the end of the sheath (or component thereof) that is initially inserted into the patient, or that is closest to the patient.

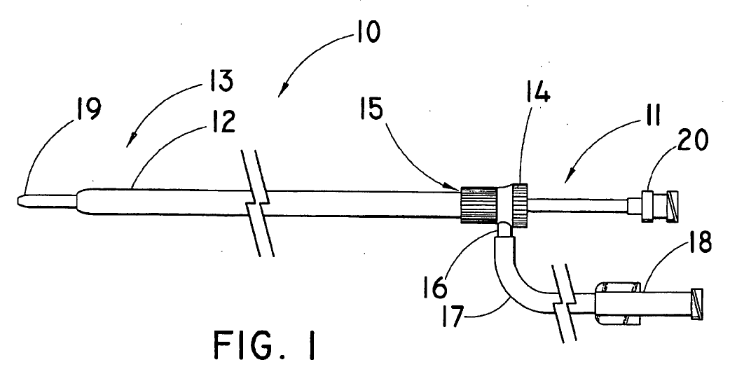

FIG. 1 illustrates one embodiment of an illustrative introducer sheath 10. Introducer sheaths are typically used in the medical field to introduce interventional devices, such as balloon angioplasty catheters or stents, and/or fluids into the vasculature of the patient. Sheaths may also be used to aspirate solids, such as a thrombus or an embolus, from the vasculature. Introducer sheath 10 includes an outer tube 12, which tube is generally provided with a tapered distal end 13, and a proximal end 15.

In FIG. 1, sheath 10 is shown in combination with a tapered dilator 11 and a connector valve 14. Dilator 11 extends longitudinally through the inner passageway of the sheath, and includes a tapered distal end 19 for accessing and dilating a vascular access site over a wire guide. Connector valve 14 is attached about the proximal end of the sheath, and generally includes one or more elastomeric disks (not shown) for preventing the backflow of fluids therethrough. The disks generally include a slit for passage of the dilator therethrough in well-known fashion. Connector valve 14 generally includes a side arm 16 to which tube 17 and male Luer lock connector 18 may be connected for introducing and aspirating fluids and/or solids through the sheath. A conventional male Luer lock connector hub 20 is attached at the proximal end of the dilator for connection to syringes and other medical apparatus. Sheaths of this general configuration are known in the medical arts, and have been disclosed, e.g., in the incorporated-by-reference U.S. Pat. No. 5,380,304.

The present invention discloses an introducer sheath that provides enhanced torqueability and has a low overall profile. In one particularly preferred embodiment, the introducer sheath also provides enhanced kink resistance. In this preferred embodiment, the invention comprises a multi-layer sheath having an inner liner, a coil wound or otherwise fitted around the inner liner, and an outer layer that comprises a torque control element.

Preferably, the torque control feature is incorporated into the outer layer by a process known in the tube extrusion art as “stripe tubing”. Stripe tubing is a well-known technique in the medical arts, and is presently used for the manufacture of devices such as feeding tubes, drainage catheters, and the like. Such devices are extruded in a manner such that a main tubular body, generally formed of a clear, transparent compound, is co-extruded with a second compound that forms one or more “stripes” disposed along the length of the main tube body. The stripes may be formed of the same or a similar base material as the main tube, and are provided to add an additional feature or utility to the device. One example of the use of such stripes is as an X-ray opacifier. In this case, the polymer comprising the stripes is formulated with an opacifier such as bismuth, barium etc. As a result of the incorporation of a radiopaque stripes in the extruded tubular body, the location of the catheter can be visualized radiographically.

In the present invention, “stripe extrusion” or “stripe tubing” technology has been expanded to incorporate one or more materials capable of being oriented to form a braid-like configuration in a layer of a catheter or sheath. The material that forms the braid-like configuration can comprise, for example, monofilament or fiber extrudable material, such as fiberglass strands, Kevlar® strands, thin wire strands, as well as filaments of conventional polymeric materials commonly used as outer layers in sheaths, such as polyamides (nylon) and polyether block amides.

In a preferred embodiment, an outer layer of a sheath is formed from two thin coaxial sleeves, each of which incorporates a helical, or twisted, stripe. The thin sleeves are coaxially aligned in a manner such that the respective pitches of the stripes are in opposite directions (see, e.g., FIG. 4). During formation of the sheath, these thin sleeves are melted in a manner such that the coaxial helical stripes are superposed, one upon another, to comprise a configuration in the nature of a braid. This configuration becomes a permanent structure that is incorporated into the outer layer of the sheath. The formation of the braid-like configuration will be further described with reference to the figures.

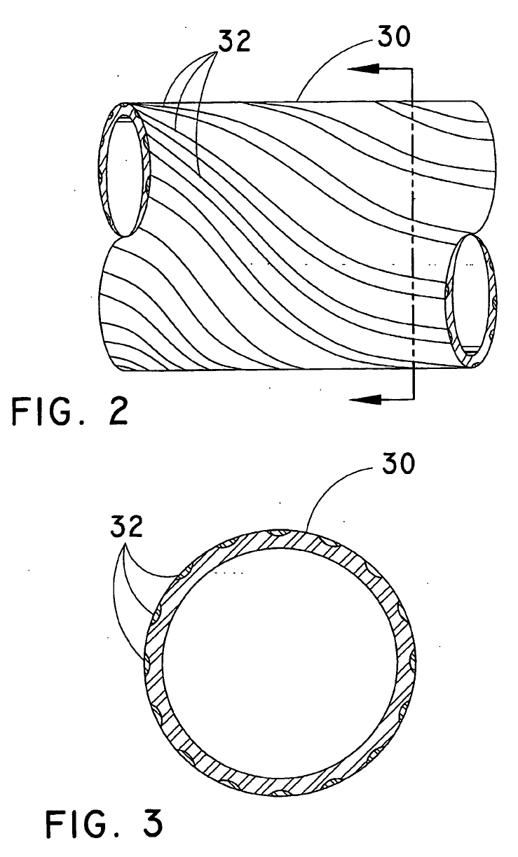

FIG. 2 is an elevational view of a sleeve 30 of the type that may be used in the formation of outer tube 12. FIG. 3 is a sectional view of tube 30, taken along line 3-3 of FIG. 2. Sleeve 30 includes a plurality of stripes 32 aligned in a helical twist pattern. The twist pattern can be formed during the stripe extrusion process by known processes. For example, the extrusion die can be biased during extrusion so that the extrudate is caused to twist as it comes out of the die. Alternatively, the twist pattern can be imparted to the tube after extrusion by twisting the tube to the desired helical pitch, and then heat setting the tube so that the twist remains.

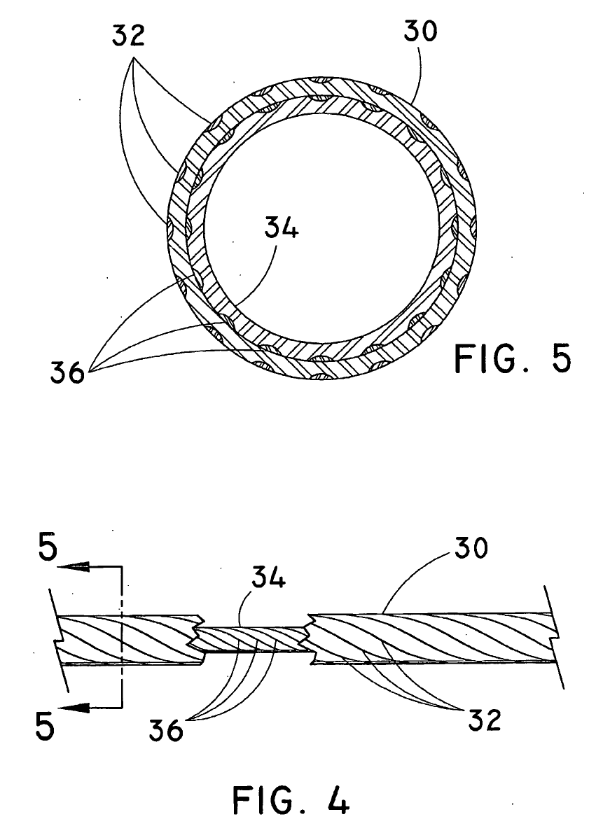

FIG. 4 is an elevational view showing sleeve 30, and further showing an inner sleeve 34 coaxially disposed within the lumen of sleeve 30. FIG. 5 is a sectional view taken along line 5-5 of FIG. 4. Inner sleeve 34 also includes a plurality of stripes 36 aligned in a helical twist pattern in the same manner as sleeve 30, but having an opposite pitch. Outer sleeve 30 and inner sleeve 34 are melted together to form outer tube 12, in a manner to be described.

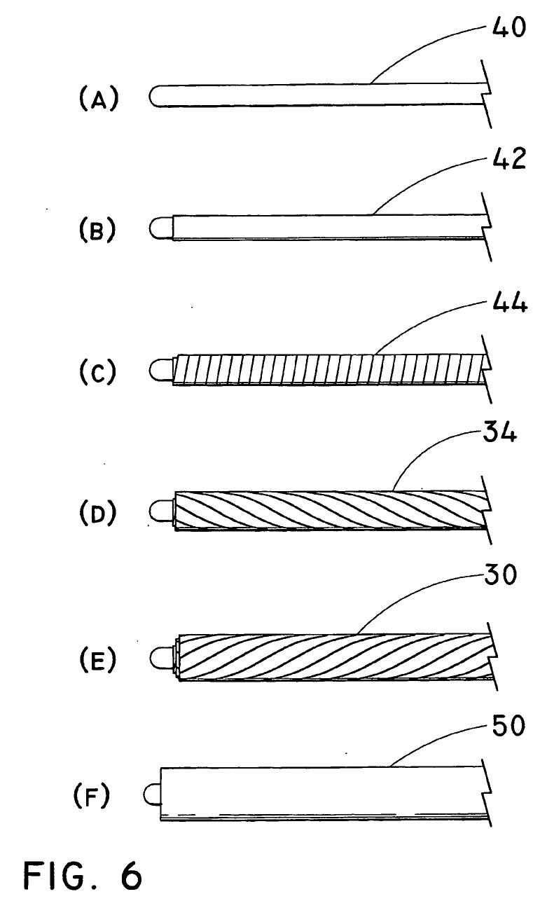

The features of the introducer sheath of the present invention, and a preferred manner of making the sheath, may be better understood upon viewing FIG. 6. FIG. 6 illustrates certain steps involved in the preparation of an introducer sheath according to one embodiment of the present invention.

Initially, a mandrel 40 is provided, as shown in FIG. 6(a). An inner liner 42 is then fitted over mandrel 40 and pulled tight onto the mandrel, as shown in FIG. 6(b). Preferably, the inner liner is a low friction, lubricious compound such as PTFE. A ribbon coil 44 is then positioned over inner liner 42, as shown in FIG. 6(c). Coil 44 may be positioned over the liner by any means known in the art, such as by compression fitting or winding the wire around the inner liner. Ribbon coil 44 is preferably stainless steel, but can be any of the materials commonly used as a coil reinforcement in the medical arts. It is preferred that the wire is flat wire to reduce the profile of the coil, but other wire cross sections, such as round wire, may be substituted if desired.

An inner striped extrusion sleeve, such as sleeve 34 in FIGS. 4 and 5, is positioned over coil 44 as shown in FIG. 6(d). An outer striped extrusion sleeve, such as sleeve 30 shown in FIGS. 2-5, is then positioned over inner sleeve 34, as shown in FIG. 6(e). Preferably, sleeves 30, 34 are formed from a polyamide material, such as nylon, or a polyether block amide. Alternatively, sleeves 30, 34 can be formed of other compounds known in the medical arts for forming sheaths, such as polyethylene, polyurethane, etc. Finally, as shown in FIG. 6(f), the entire assembly is enveloped in an outer heat shrink tube 50, formed of a known heat-shrinkable material such as PTFE or FEP.

Following arrangement of the components as described, the heat shrink tube containing the sheath assembly is then baked in an oven at a sufficient temperature and for a sufficient time to cause the heat shrink tube 50 to shrink, and to cause the outer nylon extrusion sleeves 30, 34 to melt. Heat shrink operations are well known in the medical arts, and those skilled in the art can readily determine appropriate heating conditions for a particular application. The melted nylon is squeezed through the coils by heat shrink tube 50, whereupon it bonds to the inner PTFE liner. Following bonding of the nylon to the inner liner, the heat shrink tube is slit open, and the heat shrink tube and the mandrel are removed from the assembly.

Various extrudable materials may be used as the stripe material, as long as they meet certain criteria. For example, the material must be compatible with the polymer comprising the outer sleeve. In addition, the material should be continuously extrudable with the outer sleeve polymer to form the striped configuration. Further, the extrudable material should have sufficient tensile strength to provide torque control when combined into a braid-like superposed configuration according to the process described above. Finally, the extrudable material will preferably have sufficient elasticity such that it can negotiate tortuous bends in the vasculature.

The sleeve may therefore be extruded with a fiber or strand of a similar material, or even a different material altogether. For example, a fiber or strand of a material such as Kevlar®, fiberglass or wire may be co-extruded, as previously stated. During extrusion of the dual sleeve layers, the two layers may be twisted in opposite directions to form contrasting helixes, and laminated together to essentially result in a braid-like configuration. Elasticity of the strip is not necessarily required, since braided configurations generally have sufficient flexibility to permit at least some bending. An elastic stripe material may be useful, however, in situations where a very soft flexible catheter is required, such as gastrojejunostomy catheters. Such catheters are inserted through the abdominal wall, maneuvered into the jejunum, then left indwelling for several weeks while they are used for feeding.

Since this is preferably a continuous extrusion process, the sheaths and catheters will normally have the stripe/braid extending all the way along the tube to the distal tip. The durometer or stiffness of the stripe material could be selected such that the distal tip of the catheter could be pre-curved, yet still provide torque transmission all the way to the distal end through the curve. Current torque control catheters normally end the braid just proximal to the curve, because metal braids result in a stiff distal tip that cannot easily be curved into the complex shapes normally used, do not allow tip tapers to be formed, and can result in the ends of the braid wire fraying or otherwise protruding from the catheter surface.

When sleeves 30, 34 are formed from nylon, a higher durometer and/or axially stretched nylon monofilament will preferably be utilized as the stripe material. In this case, the nylon stripe material is compatible with the nylon sleeve material, and is well incorporated into the extrusion. The nylon stripe material has sufficient tensile strength to provide favorable torque control, and yet retains sufficient elasticity to enable the tube to negotiate a tight radius bend. One non-limiting example of a suitable combination of materials comprises the use of a 50 durometer nylon material as the main sleeve body, and a 90 durometer nylon stripe material. In this case, the higher durometer stripe material provides additional torque transmission to the sleeve. Those skilled in the art can readily select an appropriate combination of materials and/or durometers for a particular application in accordance with the teachings provided herein.

Preferably, sleeves 30, 34 have very thin walls (e.g. 0.005 to 0.010 inch) [0.127 to 0.254 mm] so as not to add undue bulk to the sheath assembly. Once the assembly has melted and sleeves 30, 34 are squeezed together, the opposite helixes of the sleeves are meshed together, such that the stripes of sleeve 30 in FIG. 6(e) are superposed over the stripes of sleeve 34 in FIG. 6(d). This superpositioning of one stripe over another stripe having an opposite pitch essentially results in the formation of a braid-like configuration by the respective stripes. This braid-like configuration of the stripes provides torque control to the polymeric layer similar to that provided by a conventional braid. Suitable combinations of tubular material and stripe material can be selected to provide a finished sheath/guide catheter having torque and kink resistance qualities to suit the particular application or anatomy targeted by the introducer sheath. In general, stripe elements having a low durometer or high elasticity are preferred in applications where the introducer sheath must negotiate tortuous pathways, such as the type encountered in the distal arteries.

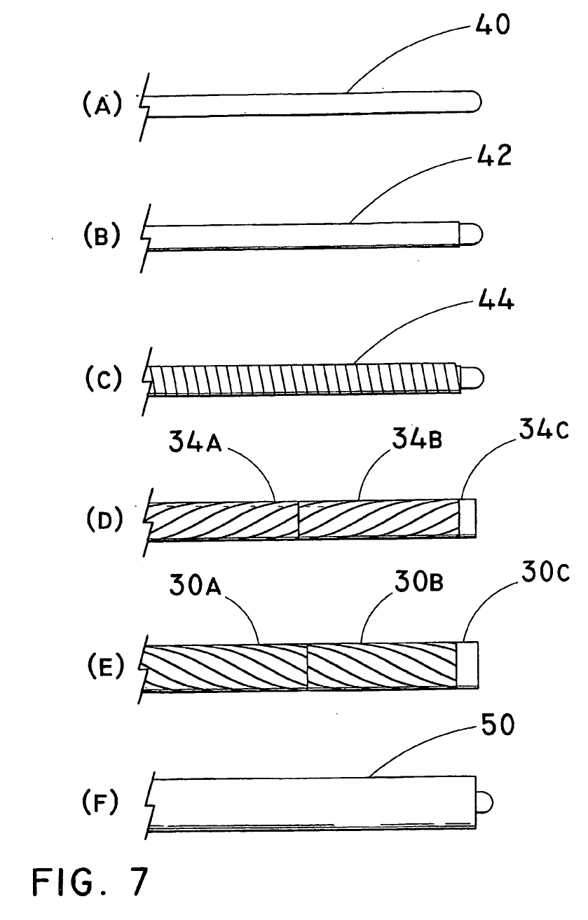

FIG. 7 shows an alternative embodiment of the present invention wherein outer sleeves 30, 34 are formed from outer sleeve segments 30a, 30b and 34a, 34b, respectively. The respective outer sleeve segments can comprise portions of differing materials, durometers and/or flexibilities. For example, proximal segments 30a, 34a can be formed of a material of a first durometer, and having a defined hardness or flexibility. Distal segments 30b, 34b can be formed of a material of a second durometer, different from the first durometer. In this way, sleeves 30, 34 can be formed such that they vary in hardness or flexibility along the length of the sleeve. In some cases, it may be desirable to make one end of the sleeve, such as the distal end, softer and more flexible than the proximal end, to enable the distal end to negotiate tight bends in the vasculature. At the same time, the proximal end can be made harder and less flexible to provide increased strength at this portion of the sheath.

Respective steps 7(a)-7(f) shown in FIG. 7 are therefore generally similar to steps 6(a)-6(f) of FIG. 6, except that steps 7(d) and 7(e) show outer sleeves 30, 34 as made up of outer sleeve segments 30a, 30b and 34a, 34b, respectively. In the embodiment shown, outer segment 34b may be positioned following the positioning of segment 34a in step 7(d), and outer segment 30b may be positioned following the positioning of segment 30a in step 7(e).

The embodiment of FIG. 7 also shows the presence of optional sleeve ends 30c, 34c. When present, sleeve ends 30c, 34c can be positioned following the positioning of segments 30b, 34b, respectively. Sleeve ends 30c, 34c can be provided when it is desired to have a discrete end portion having different properties from the remainder of the sleeve. One example of a property that may be desirable is to form distal tips 30c, 34c to be highly radiopaque. The presence of radiopaque ends enables the operator to visualize the precise distal end of the resulting tube under radiography.

Although the embodiment of FIG. 7 includes sleeve segments 30a, 30b, 30c, and 34a, 34b, 34c, respectively, the invention is not so limited. Rather, the outer sleeves may have any number of segments that can be arranged to provide desired features to the sheath. Further, the respective segments can be aligned in any desired manner to provide desired any features, such as a range of decreasing durometers, that may be desired for a particular application.

In addition to the foregoing, the formation of a polymeric braid-like configuration and the continuous extrusion of the stripes with a polymeric tube provides another benefit over prior art tubes that include metallic braids. Normally, when a metallic braid is used, the axial ends of the braid are difficult to control during the assembly and baking stages, and the ends of the wires of the braid are prone to form sharp ends that protrude from the surface of the finished device. When a braid-like configuration is formed from extruded polymers as described, the axial ends of the extruded tube do not include sharp or frayed ends. In addition, if desired, the extrusion process can even be further controlled to restrict the “stripes” to defined portions of the extruded tube. In this manner, stripes can be omitted from the axial ends, as well as any other portions of the tube in which they may not be desired or beneficial to the particular application.

The embodiment shown in FIGS. 2-5 illustrates tubes with stripe extrusions incorporated on the outside diameter of the sleeves. However, this placement of the stripe extrusions is not critical, and the stripes could alternatively be extruded to be positioned in the central part of the wall or at the inside diameter. As a still further alternative, the stripe extrusion on one of the sleeves, such as the outer sleeve, can be provided on the inner surface of this sleeve, and the stripe extrusion on the other sleeve, such as the innermost sleeve, can be provided on the outer surface of this sleeve. In this way, the stripes could be positioned to be in intimate contact following melting of the sleeves to provide a braid-like configuration that more closely resembles a braid of conventional wire or monofilament braid construction.

Although the inventive sheath has been described above as including both a coil and a braid-like structure, the invention is not so limited. Rather, it is not necessary to include the coil in all embodiments, and if desired, the coil can be omitted altogether. Among other uses, this embodiment may find particular application when the kink resistant capability of the coil is not deemed necessary for the application as hand. In this embodiment, the sheath can comprise an inner liner and an outer layer including the braid-like configuration as described. The method for making the sheath, described above, would of course be altered to omit the step relating to the positioning of the coil over the liner.

As a still further variant of the invention, the liner can also be omitted if desired. In this event, the sheath can comprise an outer jacket including the braid-like configuration as described, with or without a coil. The method for making the sheath would be altered such that the innermost sleeve may be positioned directly on a mandrel or a related type of supporting structure.

The tubular construction of the present invention also lends itself well to the construction of micro-catheters. Such catheters are similar to conventional introducer sheaths except that they are very small in diameter (below 3 or 4 French) [below 1 or 1.35 mm], and are very long so that they can reach distal arteries, such as the arteries in the brain (100 cm or more). Small diameter sheaths are normally more kink resistant than larger diameter sheaths. As a result, the embodiments wherein the coil has been omitted may be particularly useful in such sheaths.

As a result of the present invention, a low profile sheath is provided wherein the torque control feature is incorporated into the outer tube of the sheath, without adding to the thickness or bulk of the device. The sheath can also be provided with a coil for added kink resistance. In addition, the problems encountered with existing braided sheaths in trying to fuse the outer layer with the wires and the inner liner, and with the fraying of the braid wires, are avoided.

It is therefore intended that the foregoing detailed description be regarded as illustrative rather than limiting, and that it be understood that it is the following claims, including all equivalents, that are intended to define the spirit and scope of this invention.

1. A method of manufacturing an introducer sheath, comprising:
positioning a first polymeric sleeve over a mandrel, the first polymeric sleeve comprising a first striped extrusion arranged in a generally helical pattern along the first sleeve;
positioning a second polymeric sleeve over the first sleeve, the second polymeric sleeve comprising a second striped extrusion arranged in a generally helical pattern along the second sleeve, the first and second polymeric sleeves being axially aligned such that said second striped extrusion is superposed over said first striped extrusion to define a generally braid-like configuration; and
heating the first and second polymeric sleeves.

2. The method of claim 1, wherein said first striped extrusion comprises a plurality of extruded first stripes formed in said first polymeric sleeve, each said first stripe spaced from an adjoining stripe and arranged in said generally helical pattern, and said second striped extrusion comprises a plurality of extruded second stripes formed in said second polymeric sleeve, each said second stripe spaced from an adjoining stripe and arranged in said generally helical pattern.

3. The method of claim 1, wherein at least one of said first and second striped extrusions is provided along an outer surface of the respective first and second polymeric sleeves.

4. The method of claim 1, wherein at least one of said first and second striped extrusions is provided along an inner surface of the respective first and second polymeric sleeves.

5. The method of claim 1, wherein the first striped extrusion is provided along an outer surface of the circumference of the first polymeric sleeve, and the second striped extrusion is provided along an inner surface of the second polymeric sleeve.

6. The method of claim 1, wherein the first polymeric sleeve is co-extruded with the first striped extrusion, and the second polymeric sleeve is co-extruded with the second striped extrusion.

7. The method of claim 1, comprising positioning an inner liner over the mandrel intermediate the mandrel and the first polymeric sleeve.

8. The method of claim 7, comprising:
positioning a coil over the inner liner, the coil having a plurality of coil turns; and
bonding the first polymeric sleeve to the inner liner between the coil turns by the heating.

9. The method of claim 8, comprising:
positioning a heat shrink tube over the assembly comprising the mandrel, inner liner, coil, and first and second sleeves;
carrying out the heating step in the heat shrink tube in a manner such that the first and second striped extrusions maintain the braided configuration; and
removing the sheath from the mandrel and the heat shrink tube.

10. The method of claim 1, wherein at least one of said polymeric sleeves comprises at least two sleeve segments.

11. An introducer sheath, comprising:
a first polymeric sleeve comprising a first striped extrusion arranged in a generally helical pattern along the first sleeve; and
a second polymeric sleeve positioned over said first polymeric sleeve and bonded thereto, said second polymeric sleeve comprising a second striped extrusion arranged in a generally helical pattern along the second sleeve, the first and second polymeric sleeves being axially aligned such that said second striped extrusion is superposed over said first striped extrusion to define a generally braid-like configuration.

12. The introducer sheath of claim 11, the first striped extrusion comprising a plurality of extruded spaced apart first stripes formed in the first polymeric sleeve and arranged in the generally helical pattern, and the second striped extrusion comprising a plurality of extruded spaced apart second stripes formed in the second polymeric sleeve and arranged in the generally helical pattern.

13. The introducer sheath of claim 11, the first and second polymeric sleeves being coaxially aligned in a manner such that a pitch of the first striped extrusion is aligned in an opposite direction from a pitch of the second striped extrusion.

14. The introducer sheath of claim 11, further comprising an inner liner disposed with a lumen of the first polymeric sleeve and bonded thereto.

15. The introducer sheath of claim 14, further comprising a coil fitted over said inner liner, said coil having a plurality of coil turns extending longitudinally around said inner liner, said first polymeric sleeve bonded to said inner liner between turns of said coil.

16. The introducer sheath of claim 11, wherein at least one of said first and second polymeric sleeves is formed from a polymer selected from the group consisting of polyamides, polyether block amides, polyethylene, polyurethane and mixtures of the foregoing.

17. The introducer sheath of claim 11, wherein at least one of said first and second striped extrusions is formed from a polymer selected from the group consisting of polyamides, polyether block amides, polyethylene, polyurethane, fiberglass strands and thin wire strands.

18. The introducer sheath of claim 16, wherein at least one of said first and second striped extrusions is formed from the same polymer that forms said respective first and second polymeric sleeve.

19. The introducer sheath of claim 11, wherein at least one of said polymeric sleeves comprises at least two sleeve segments.

20. The introducer sheath of claim 19, wherein each of said polymeric sleeves comprises a plurality of sleeve segments, said sleeve segments in each said polymeric sleeve being arranged in order of decreasing durometer from a proximal end to a distal end of said polymeric sleeve.
